(19) United States
(12) Patent Application Publication
(10) Pub. No.: US 2025/0090266 A1
(43) Pub. Date: Mar. 20, 2025

(54) ROTATING MARKER AND ADAPTER FOR IMAGE-GUIDED SURGERY

(71) Applicant: AUGMEDICS LTD., Yokneam Illit (IL)

(72) Inventors: Tomer Gera, Kfar-Tavor (IL); Nissan Elimelech, Beerotaim (IL)

(21) Appl. No.: 18/291,731

(22) PCT Filed: Jul. 28, 2022
(86) PCT No.: PCT/IB2022/056986

§ 371 Date: Jan. 24, 2024

Related U.S. Application Data
(63) Continuation-in-part of application No. 17/388,064, filed on Jul. 29, 2021, now Pat. No. 12,150,821.

(51) Int. Cl. A61B 90/00; A61B 34/20

(52) U.S. Cl. CPC A61B 90/39; A61B 34/20; A61B 90/361; A61B 2034/2055; A61B 2090/3983; A61B 2090/3991

(57) ABSTRACT

A patient marker (52) couples to an anchoring device (34) via a base (44) having a base axis, base connections and a first indicator (168). The marker includes: an adapter (40) having a first surface with connections configured to mate with the base connections, and a second surface with connections congruent with the base connections, and at least one second indicator (172). The marker includes an alignment target (48), having a target region (74) with an alignment pattern, and a socket (78) connected to the target region with socket connections congruent with the first surface connections. In a first configuration the socket couples to the base by mating the first surface connections with the base connections and mating the socket connections with the second surface connections. In a second configuration, the socket fits onto the base by mating the socket connections with the base connections.

### FIELD

The present disclosure relates generally to a marker and a marker adapter for image-guided surgery, and particularly to a marker that may be attached to a patient and/or to a marker adapter that allows multiple orientations of a marker.

### BACKGROUND

During image guided surgery, it is important to register elements of a patient, upon whom the surgery is being performed, with equipment generating the image. This is typically the case where the surgery comprises an augmented reality system, which generates images of portions of the patient that are in registration with the actual portions.

In order to register the elements of the patient, and keep the registration, a patient marker may be fixedly attached to the patient. An initial image of a marker may be analyzed, and used to provide the registration. So long as a marker continues to be fixedly attached to the patient at the same location, the registration remains valid and may be used to track the patient. However, if the marker is moved and then re-attached, for example to provide access to the patient, the registration typically needs to be repeated.

Documents incorporated by reference in the present patent application are to be considered an integral part of the application except that, to the extent that any terms are defined in these incorporated documents in a manner that conflicts with definitions made explicitly or implicitly in the present specification, only the definitions in the present specification should be considered.

### SUMMARY

An embodiment of the present disclosure provides a patient marker for image guided surgery that is configured to be coupled to an anchoring device via a base, the base having a base axis, base connections and a first indicator, the patient marker including:
an adapter having a first surface having first surface adapter connections configured to mate with the base connections, and a second surface, opposite the first surface, having second surface adapter connections congruent with the base connections, and at least one second indicator; and
an alignment target, including:
a target region having an alignment pattern formed thereon; and
a socket connected to the target region and having socket connections congruent with the first surface adapter connections, so that:
in a first configuration of the marker, the socket is coupled to the base by mating the first surface adapter connections with the base connections and mating the socket connections with the second surface adapter connections, and
in a second configuration of the marker, the socket is fit onto the base by mating the socket connections with the base connections,
whereby an angle of orientation of the alignment target about the base axis is indicated by one of the first indicator and the at least one second indicator.

Typically, in any given configuration of the marker, only one of the first indicator and the at least one second indicator is accessible.

In a disclosed embodiment in the first configuration the alignment target fits to the adapter in a plurality of discrete orientations. The discrete orientations may consist of two orientations at 180° to each other.

In a further disclosed embodiment in the second configuration the alignment target fits to the base in a plurality of discrete orientations. The discrete orientations may consist of two orientations at 180° to each other.

In a yet further disclosed embodiment in the first configuration the angle of orientation includes a first pair of angles at 180° to each other, and in the second configuration the angle of orientation includes a second pair of angles at 180° to each other and at 90° to the first pair of angles.

In an alternative embodiment the base connections and the first surface adapter connections are configured so that the adapter mates with the base in one single orientation.

In a further alternative embodiment the at least one second indicator is a divot that acts as a verification point.

In a yet further alternative embodiment the at least one second indicator is formed as an extension of a surface of the adapter.

In a still further alternative embodiment one of the at least one second indicator is positioned so that it aligns with and prevents access to the first indicator, while the first surface adapter connections are mated with the base connections.

The at least one second indicator may consist of two indicators. The angle between lines from each of the two indicators to a central hole of the adapter may be obtuse.

The at least one second indicator may be configured to be touched by a tip of a tool used in the surgery.

There is further provided, according to an embodiment of the present disclosure, a method for producing a patient marker to be coupled to an anchoring device via a base, the base having a base axis, base connections and a first indicator, the method including:
configuring first surface adapter connections of an adapter having a first surface to mate with the base connections;
forming on a second surface of the adapter, opposite the first surface, second surface adapter connections congruent with the base connections, and at least one second indicator; and
providing an alignment target, consisting of:
a target region having an alignment pattern formed thereon, and
a socket connected to the target region and having socket connections congruent with the first surface connections, so that:
in a first configuration of the marker, the socket is coupled to the base by mating the first surface adapter connections with the base connections and mating the socket connections with the second surface adapter connections, and
in a second configuration of the marker, the socket is fit onto the base by mating the socket connections with the base connections,
whereby an angle of orientation of the alignment target about the base axis is indicated by one of the first indicator and the at least one second indicator.

There is further provided, according to an embodiment of the present disclosure, an adapter for providing additional orientations to a patient marker with respect to an anchoring device in image guided surgery, wherein the anchoring device has a base having a base axis and a first indicator, the adapter including:
a first surface configured to be coupled to the base;
a second surface, opposite the first surface, configured to be coupled to the patient marker; and
at least one second indicator,
wherein the patient marker is coupled to the base by coupling the first surface of the adapter with the base and coupling the patient marker with the second surface of the adapter, and wherein when the patient marker is coupled to the anchoring device via the adapter:
an angle of orientation of the patient marker about the base axis is indicated by only one of the at least one second indicator, and
the first indicator is not accessible.

Typically, the at least one second indicator consists of two indicators, and wherein when the patient marker is coupled to the anchoring device via the adapter, only one of the two indicators is accessible. Additionally or alternatively, the at least one second indicator may be configured to be touched by a tip of a tool used in the surgery and serve as a verification point.

There is further provided, in accordance with yet another embodiments of the disclosure, a computerized method for updating a registration between a patient marker and a patient due to a change in the orientation of the patient marker during a medical procedure, wherein the patient marker is coupled to an anchoring device via a an adapter, the adapter is coupled to the anchoring device via a base, the anchoring device is attached to the patient, and the base comprises at least one base indicator, and the adapter comprises at least one adapter indicator. The method includes:
registering the patient marker to the patient;
storing a plurality of distinct orientation values, each orientation value corresponds to a different orientation of the patient marker with respect to the anchoring device and associated with an indicator, wherein the indicator is the at least one base indicator or the at least one adapter indicator, and wherein the orientation value indicates a position of the indicator associated with the orientation value with respect to the patient marker;
tracking the position of a tool marker with respect to the patient marker by capturing images of the patient marker and the tool marker via a camera, wherein the tool marker is attached to a tool used in the medical procedure;
determining a current marker orientation value based on analysis of one or more images of the patient marker and the tool marker;
repeatedly calculating subsequent marker orientation values for images captured by the camera; and
if a change in marker orientation value was identified in the subsequent marker orientations with respect to the current marker orientation value, then a change in the patient marker orientation is determined and the registration of the patient marker to the patient is updated accordingly.

In a disclosed embodiment, in any orientation of the patient marker with respect to the anchoring device, only one of the at least one base indicator or the at least one adapter indicator is accessible.

In a further disclosed embodiment, the base comprises base connections, the adapter comprises adapter connections and the patient marker comprises marker connections, and wherein the base connections are used to connect the base to both the patient marker and the adapter, in an alternative manner, and the marker connections are used to connect the patient marker to both the base and the adapter, in an alternative manner.

The present disclosure will be more fully understood from the following detailed description of the embodiments thereof, taken together with the drawings in which:

### BRIEF DESCRIPTION OF THE DRA WINGS

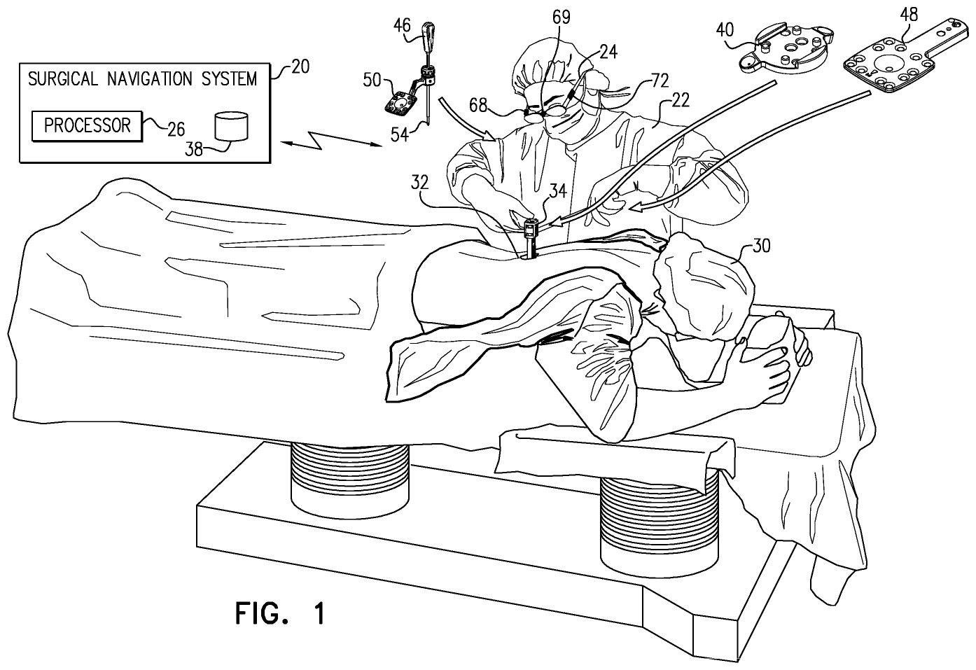

FIG. 1 is a schematic illustration of a medical procedure, according to an embodiment of the present disclosure;

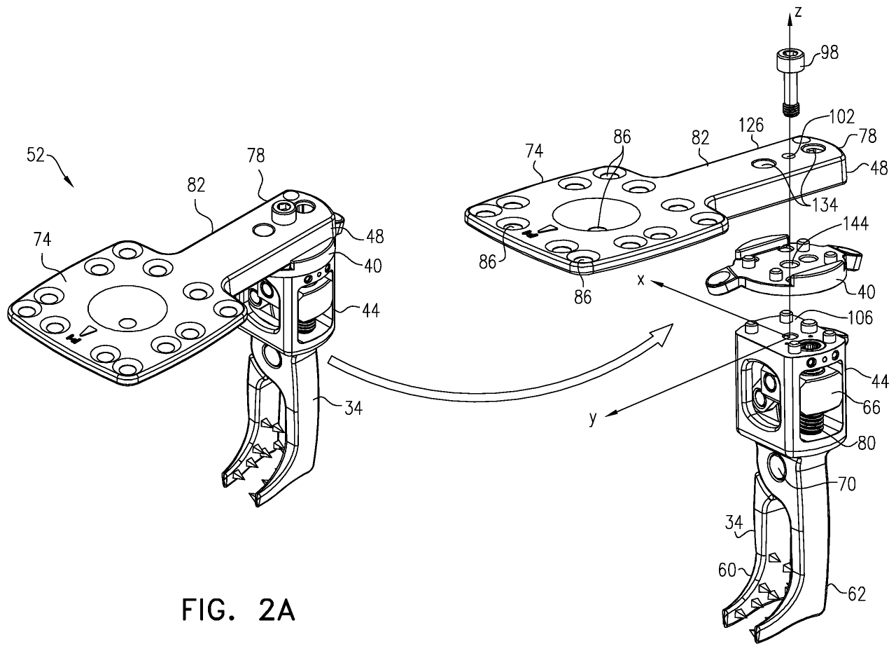

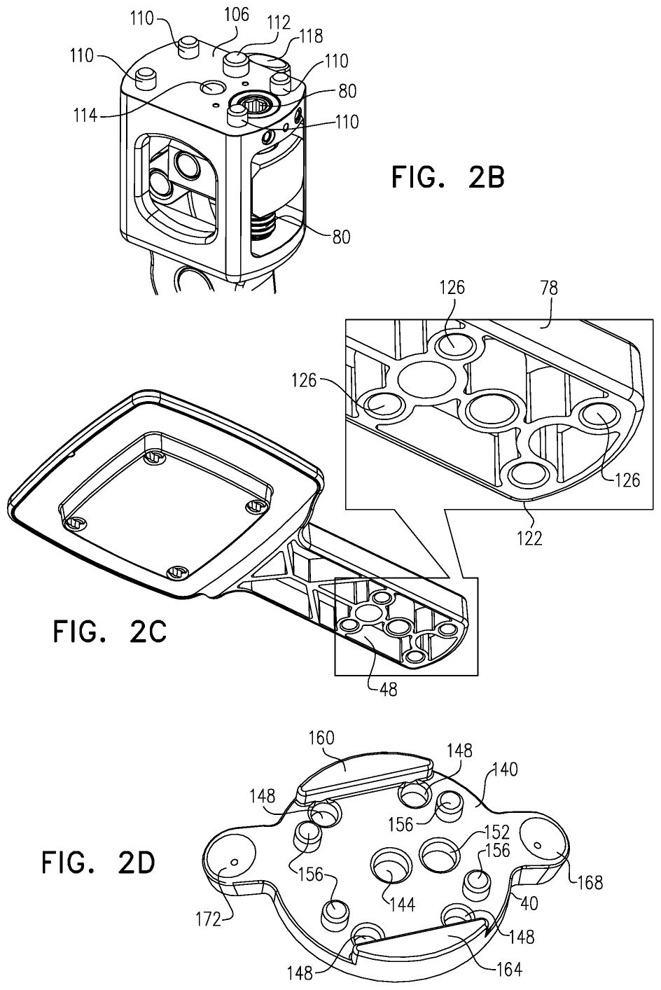

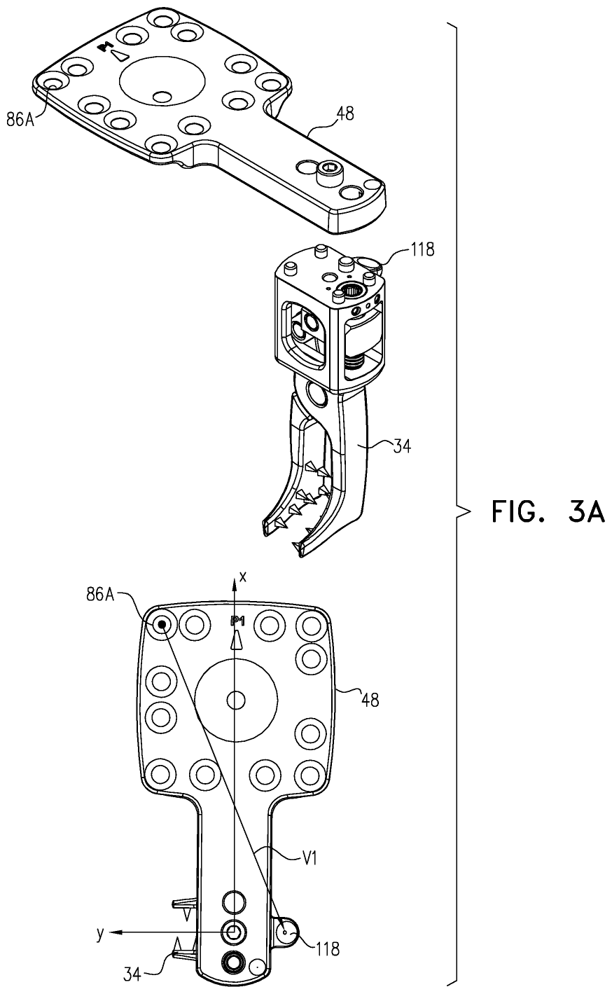

FIG. 2A illustrates elements of a patient marker or an adapter, in an assembled and a partly exploded format, and FIGS. 2B, 2C, and 2D illustrate elements of portions of the marker, a base or an adapter, according to embodiments of the present disclosure;

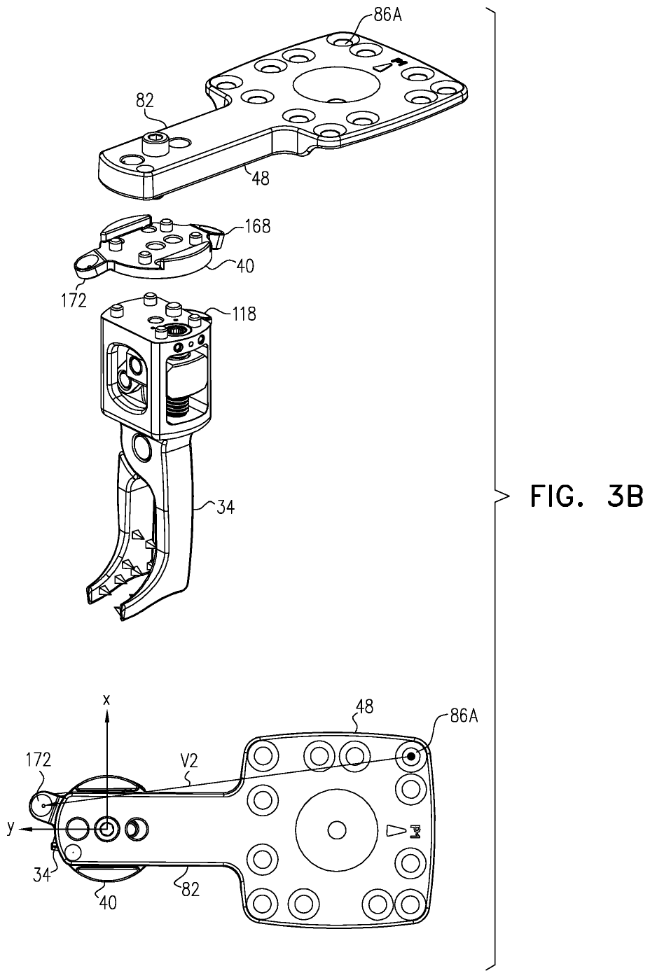

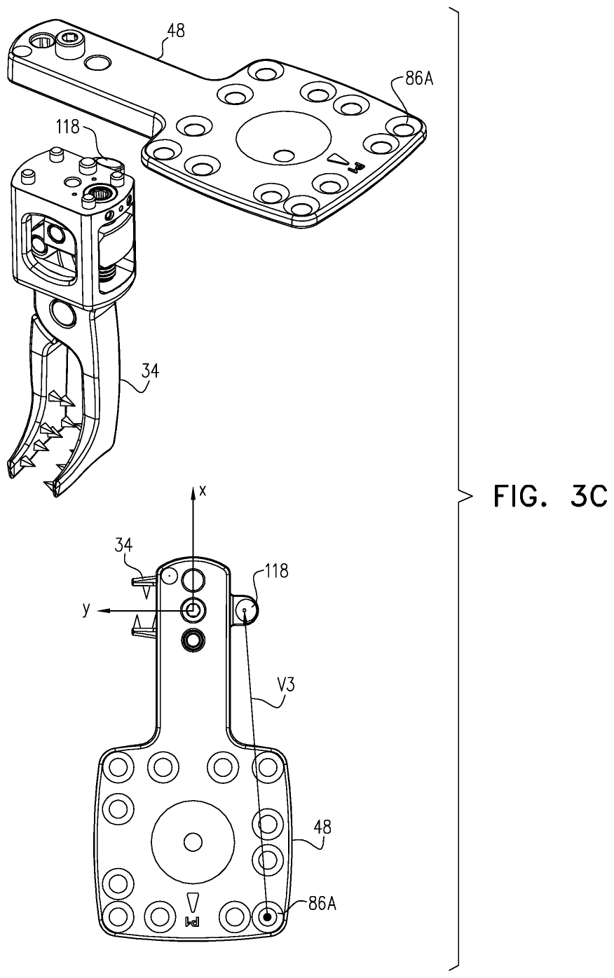

FIGS. 3A, 3B, 3C, and 3D illustrate four orientations of a target of the patient marker with respect to a clamp, according to an embodiment of the present disclosure; and

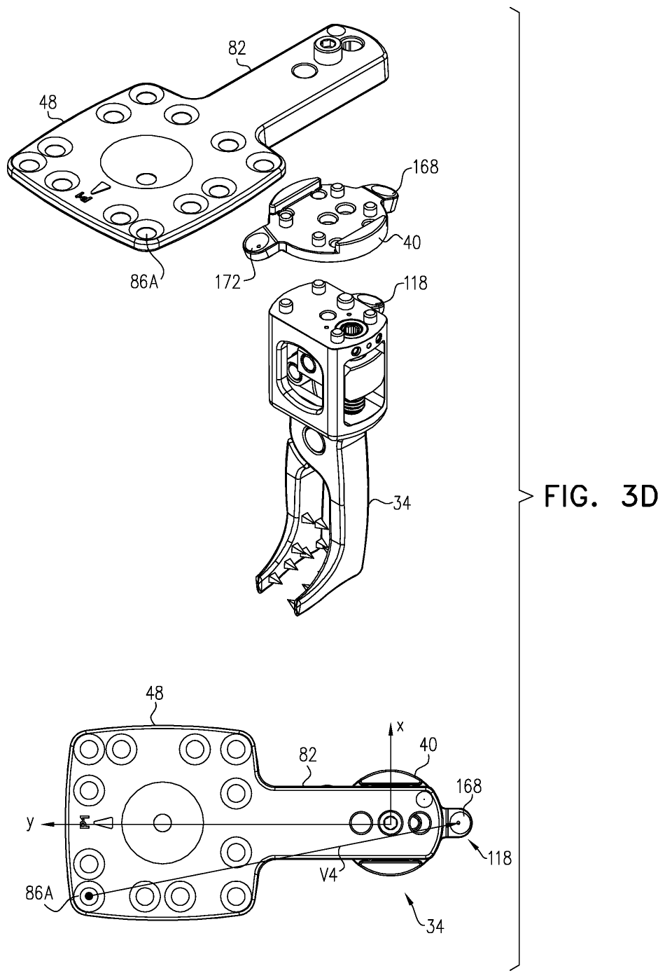

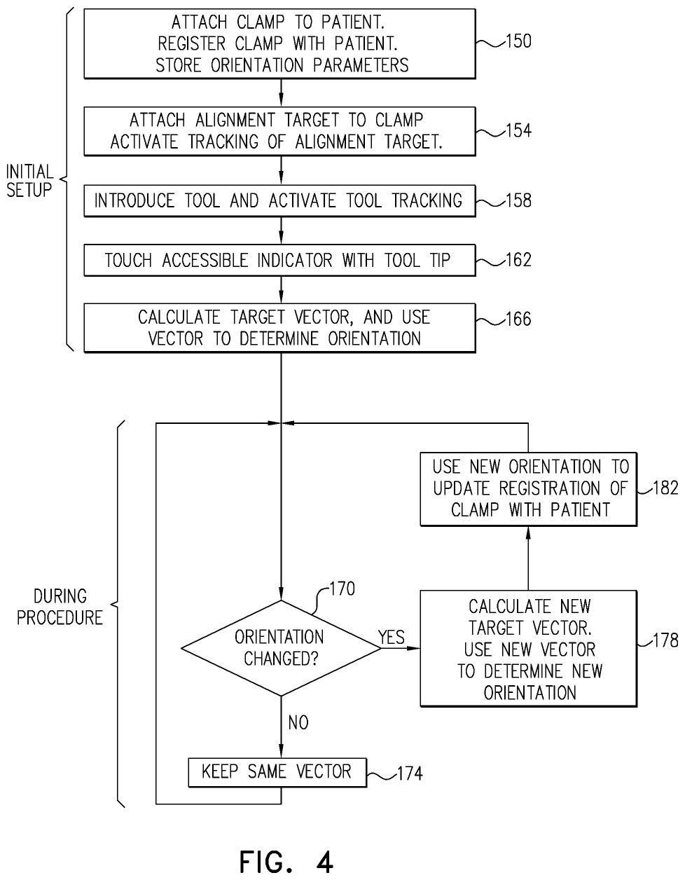

FIG. 4 is a flowchart describing the use of the marker and/or adapter in the medical procedure and a computerized method for updating a registration between a patient marker and a patient due to a change in the orientation of the patient marker during a medical procedure, according to an embodiment of the present disclosure;

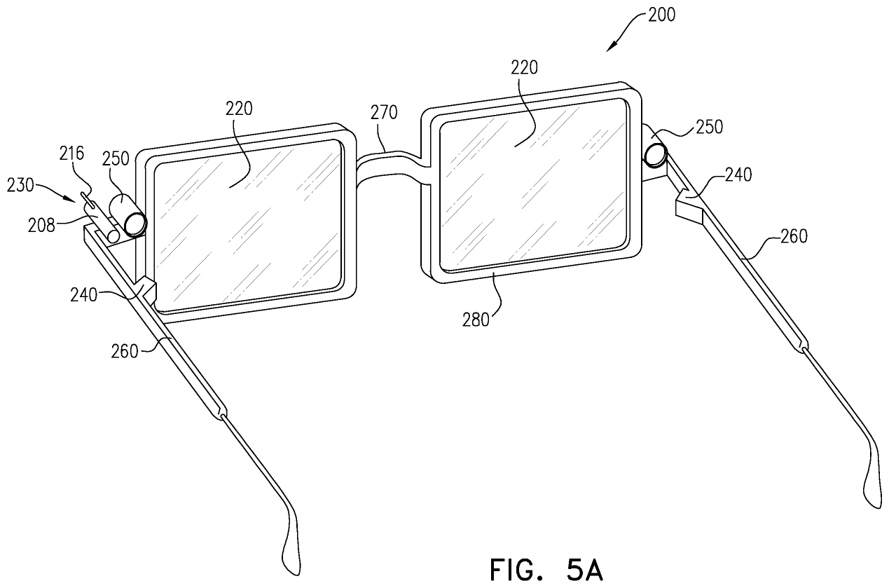

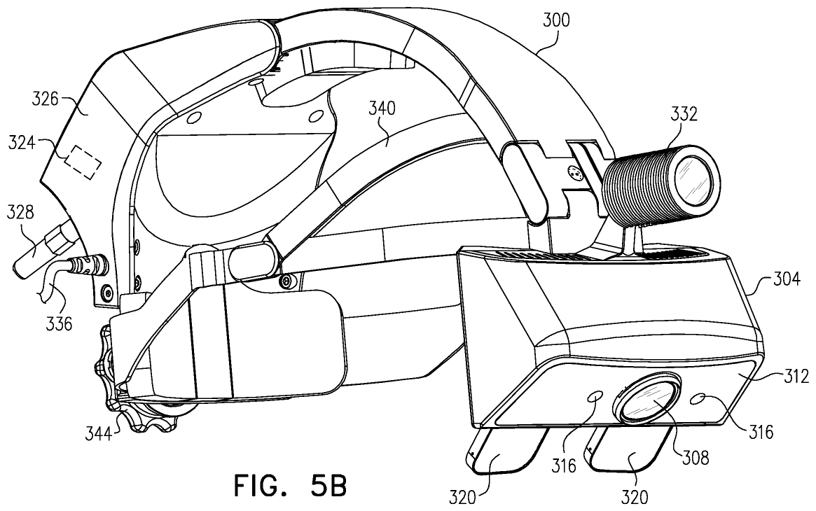

FIG. 5A is a schematic illustration of a head-mounted display (HMD), according to an embodiment of the present disclosure;

FIG. 5B is a schematic illustration of a head-mounted display (HMD), according to another embodiment of the present disclosure.

### DETAILED DESCRIPTION

### Overview

In an augmented reality (AR) system or, generally, in an image-guided system that is used for a medical procedure performed on a patient, the position of the patient, e.g., in relation to an augmented reality assembly worn by a professional performing the procedure, and/or in relation to a tool navigated within the patient body, needs to be tracked. Thus, for example, images presented in the AR assembly or on a display align with the patient and/or are presented from a certain point of view, and/or align with the tool. To implement the tracking, a patient marker, comprising optical elements, may be fixedly attached to the patient, and a camera in the assembly or of an image-guided system may be configured to image the elements. A processor may then analyze the image so as to track the marker. As long as the marker is fixed to the patient, the processor may use the tracking of the marker to track the patient.

However, during the procedure the patient marker may interfere with the professional's access to the patient, and/or the professional's view of the patient, necessitating adjustment of the patient marker.

Embodiments of the present disclosure provide a patient marker which may be adjusted to a plurality of orientations and during a medical procedure, while not requiring any new registration or re-registration and image analysis of the optical elements of a registration marker and the oriented marker to determine their new or adjusted relative position to allow the continuation of the tracking.

Although the use of disclosed embodiments is described with respect to an image-guided system employing AR (e.g., an AR system), the disclosed embodiments may be used, mutatis mutandis, with other image guided systems utilizing optical tracking.

A disclosed embodiment of the present disclosure provides a patient marker comprising a base connecting to an anchoring device such as a pin inserted to a bone or a clamp. The anchoring device herein by way of example assumed to comprise a clamp. Anchoring devices of this sort for image-guided surgery are described, for example, in PCT International Publication WO 2022/079565 and PCT International Application IB2022/056212. The disclosures of all these patent applications and publications are incorporated herein by reference. The clamp has jaws configured to grip a bone of the patient, such as a spinous process. Once the jaws have gripped the bone of the patient, the base provides a rigid platform to which may be attached an alignment target that does not move relative to the patient's bone.

The alignment target may be attached, in a first configuration of the marker, directly to the base in one of a first plurality of preselected orientations. In some embodiments, the alignment target may be attached in a first configuration of the marker directly to the base in a single preselected or predetermined orientation. The term “plurality of configuration” may relate hereinafter to one or more configurations, according to the term context. An image of the alignment target may then be acquired by the camera in the augmented reality assembly worn by the professional, e.g., to implement tracking of the target and of the patient. The target may be adjusted between the first plurality of preselected orientations, without requiring any new registration and any image analysis by the processor of the imaged adjusted target and the registration marker, so that the processor may continue the tracking.

To provide further orientations of the target, embodiments of the disclosure provide an adapter. Rather than attaching the target directly to the base, as in the first configuration described above, in a second configuration of the marker the target is attached to the adapter which is then attached to the base. The adapter provides the target with a second plurality of preselected orientations (e.g., one or more additional preselected orientation), different from the first plurality. As for the second configuration, the target may be adjusted between the second plurality of preselected orientations, without requiring any new registration or image analysis of the registration marker and the patient marker or target, so that the processor may continue the tracking. Furthermore, the target may be adjusted between the first plurality of preselected orientations and the second plurality of preselected orientations, without requiring any new registration or such image.

In addition, in some embodiments, the adapter is configured so that the first and second plurality of preselected orientations are simply related. In a disclosed embodiment the first plurality of configurations, which does not use the adapter, provides a first orientation wherein the target rotates 0° about a base axis and a second orientation wherein the target rotates 180° about the axis. The second plurality of configurations, which uses the adapter, provides a third orientation where the target rotates 90° about the axis and a fourth orientation where the target rotates 270° about the axis. Since the alignment target may be attached, with or without use of the adapter, to the base in a multiplicity of preselected orientations, embodiments of the disclosure provide a simple and cost effective solution for adjusting the patient marker and without requiring new registration and image analysis of the target of the patient marker and the registration marker.

According to some aspects, an adapter, as described above, is provided, which allows further orientations to a marker. The adapter provides additional orientations to the patient marker with respect to the anchoring device. The base (referred to above) connecting to the anchoring device, or comprised in the anchoring device, may be provided by the anchoring device, and the base has a base axis and at least a first indicator. In some embodiments, the base may comprise more than one indicator.

The adapter has two surfaces: a first surface that may be coupled to the base, e.g., by the first surface adapter connections, and a second surface, opposite the first surface, that may be coupled to the patient marker, e.g., via the second surface adapter connections. The adapter also has at least one, e.g., two, second indicators. The patient marker may be coupled to the base by coupling the first adapter surface to the base, and coupling the second adapter surface to the patient marker, so that the patient marker couples to the anchoring device via the adapter.

In each orientation, only one indicator is accessible to prevent confusion and to allow unambiguous determination of the orientation. When the patient marker is coupled to the anchoring device via the adapter, as described above, the angle of orientation of the patient marker about the base axis is indicated by only one of the second indicators. For example, in case of two adapter indicators, when the marker is coupled to the adapter, only one indicator is accessible while the other is covered by the marker (e.g., positioned beneath the marker as exemplified in FIGS. 3B and 3D). Furthermore, the first indicator is not accessible (e.g., covered by the adapter, as shown in FIGS. 3B and 3D).The adapter may be coupled to the anchoring device via its base and may be coupled to the marker; it may be formed, configured to and operate, all as detailed herein above and herein below, with the necessary changes.

During the medical procedure, in addition to tracking the patient marker, a position of the tip of a tool used in the procedure is also tracked. The base of the anchoring device has a verification point or divot fixedly located, in a known preselected position in the base. The adapter also has at least one, and herein by way of example two verification points, fixedly located in known preselected positions on the adapter.

To identify, during the procedure, which of the preselected orientations of the target is operative, the tool tip is placed on, or in proximity to, one of the verification points of the base or the adapter. The processor of the augmented reality system calculates the location of the indicated verification point with respect or relative to the tracked alignment target or patient marker, e.g., by calculating a vector between the indicated verification point and the tracked alignment target, and from that calculated location or vector (which are different for all the target orientations) identifies the orientation of the target or patient marker.

### Detailed Description

In the following, all directional references (e.g., upper, lower, upward, downward, left, right, top, bottom, above, below, vertical, and horizontal) are only used for identification purposes to aid the reader's understanding of the present disclosure, and do not create limitations, particularly as to the position, orientation, or use of embodiments of the disclosure.

Reference is now made to FIG. 1, which is a schematic illustration of a medical procedure, according to an embodiment of the present disclosure. During the procedure, performed by a professional 22, the professional uses a surgical navigation system 20, which assists the professional in performance of the procedure. Surgical navigation system 20 comprises a processor 26, which operates elements of the system, and which communicates with an augmented reality assembly 24, comprising a set of augmented reality spectacles worn by professional 22, and that is incorporated in the system. Assembly 24 comprises an imaging device. The imaging device may comprise one or more imaging devices. The imaging device is configured, inter alia, to track one or more elements, such as marker 48 or a medical tool used during the procedure. Assembly 24 comprises, for example, an image capturing device 72, also termed herein a camera 72, that has a field of view and that is configured to capture images in the visible spectrum. Assembly 24 also comprises an image capturing device 68, also termed a camera 68, that is operative in the non-visible spectrum, typically the infra-red, as well as a projector 69 that projects radiation in the operative spectrum of device 68. Functions of system 20, processor 26, and devices 68 and 72 are described below. Assemblies similar to augmented reality assembly 24, and its operation, are described in U.S. Pat. No. 9,928,629, to Benishti, et al., and PCT International Publication WO 2022/053923 whose disclosures are incorporated herein by reference.

It will be understood that the present disclosure includes augmented reality assemblies other than assembly 24 described here. Examples of such assemblies are described with reference to FIGS. 5A and 5B, and all such assemblies are assumed to be comprised within the scope of the present disclosure.

processor 26 may include one or more processing units. In one embodiment processor 26 is assumed to be incorporated within a stand-alone computer, and the processor typically communicates with other elements of the system, including assembly 24, wirelessly, as is illustrated in FIG. 1. Alternatively or additionally, processor 26 may use optical and/or conducting cables for the communication. In further alternative embodiments processor 26 is integrated within assembly 24, or in the mounting of the assembly. According to an embodiment, processor 26 includes two processing units, while one processing unit is integrated within a work station and the other is integrated within assembly 24. Processor 26 is typically able to access a database 38, wherein are stored images and other visual elements used by system 20. Software enabling processor 26 to operate system 20 may be downloaded to the processor in electronic form, over a network, for example. Alternatively or additionally, the software may be provided on non-transitory tangible media, such as optical, magnetic, or electronic storage media.

The medical procedure exemplified here is performed on a patient 30, and during an initial stage of the procedure professional 22 makes an incision 32 into the patient's back. The professional then inserts an anchoring device 34, herein assumed to comprise a spinous process clamp 34, into the incision, so that opposing jaws of the clamp are located on opposite sides of the spinous processes. The professional adjusts the clamp to grip one or more spinous processes, selected by the professional, of the patient. The professional may then attach an orientation adapter 40 to a base 44 (illustrated in FIG. 2A) of the clamp, and an alignment target 48 to the adapter, the target when attached to the base via the adapter optionally operating as a patient marker 52 (illustrated in FIG. 2A). According to some embodiments, patient marker 52 comprises alignment target 48 coupled, as described herein, to base 44. According to some embodiments, patient marker 52 comprises alignment target 48 and may be coupled directly to base 44 or via orientation adapter 40. As is described below, processor 26 tracks alignment target 48 of patient marker 52 (e.g., via camera 68), and the tracking is used by system 20 to determine the position and orientation of patient 30 during the medical procedure.

During the procedure professional 22 uses a tool 46 comprising a tool marker 50. Processor 26 also tracks tool 46 using tool marker 50 attached to the tool, and because the dimensions of the tool and the tool marker are known, the tracking provides the processor with the position of a tool tip 54 of the tool. A tool and tool marker similar to tool 46 and marker 50 are described in U.S. Patent Publication No. 10,939,977 and U.S. Application Publication No. 2022/0071712, which are incorporated herein by reference.

During the procedure, it may be necessary for professional 22 to adjust the position of target 48 or patient marker 52, typically to enable the professional to more easily access elements of patient 30. Embodiments of the present disclosure facilitate such adjustment, by providing a plurality of different discrete orientations of alignment target 48 or marker 52 with respect to clamp 34. Each of the orientations may be identified by processor 26 from an acquired image of target 48 and from the tracked position of tool tip 54, as is described below. In addition, as is described below, once patient marker 52 has been through an initial setup process to align images presented in assembly 24 with patient 30, processor 26 may maintain the image alignment for the different identified orientation changes, by virtue of the fact that dimensions of target 48, adapter 40, and/or base 44 are known.

FIG. 2A illustrates elements of patient marker 52 and adapter 40, in an assembled and a partly exploded format, and FIGS. 2B, 2C, and 2D illustrate elements of portions of the marker, the base or the adapter, according to an embodiment of the present disclosure. As stated above, marker 52 may be formed by attaching alignment target 48 to adapter 40, which is in turn attached to base 44 of an anchoring device such as clamp 34. According to aspects of the present disclosure, marker 52, which comprises alignment target 48, may be attached to adapter 40, which may be in turn attached to base 44 of clamp 34. The clamp is described below.

Clamp 34 comprises a pair of jaws 60, 62 in a lower section of the clamp. The jaws are coupled to clamp base 44 in an upper section of the clamp, the base comprising a jaw adjustment mechanism 66. In the embodiment described herein, jaw 62 is fixed to base 44, and jaw 60 moves with respect to jaw 62, by being rotated about a hinge pin 70. Jaw adjustment mechanism 66 comprises an adjustment screw 80, which is coupled by a lever to jaw 60 so that rotation of the screw causes jaw 60 to approach or retreat from jaw 62. Thus professional 22 is able to cause the jaws of clamp 34 to grip or release a bone, such as one or more spinous processes, by rotating screw 80.

Alignment target 48 comprises a target region 74 and a socket 78, the target region and the socket being fixedly connected together by a connecting beam 82. Alignment target 48, together with its components target region 74 and socket 78, are generally planar. Target region 74, by way of example, is approximately rectangular, e.g., approximately square, and comprises optical elements 86. Elements 86 are arranged in a three-dimensional (3D) pattern, although in some embodiments the elements may be in a two-dimensional (2D) pattern, with no rotational axis of symmetry (other than a trivial axis of symmetry for rotating by 360°), and no mirror plane of symmetry, so that an image of the elements enables an unambiguous determination of the location and orientation of the target region. Elements 86 are typically retroreflectors. An entity with an arrangement of optical elements similar to the arrangement herein is described in U.S. Patent Publication No. 10,939,977, which is incorporated hereinabove by reference.

For clarity, in the description herein, elements of marker 52 are assumed, by way of example, to be referenced to a Cartesian set of orthogonal xyz axes, with origin at the center of an upper plane surface 106 of base 44, as illustrated in FIG. 2A. A z-axis is assumed to be orthogonal to plane surface 106, a y-axis is assumed to be parallel to a symmetry axis of hinge 70, and an x-axis is assumed to be orthogonal to the y and z axes. As another example, the origin may be at the center of an optical element 86A of marker 52.

As stated above, socket 78 is generally planar, and has a central hole 102. As is explained below, socket 78 and adapter 40 are fixed to base 44 by a captive screw 98 that penetrates a socket central hole 102 and also an adapter central hole 144. Holes 102 and 144 are dimensioned as clearance holes for screw 98.

FIG. 2B illustrates upper surface 106 of base 44. Surface 106 comprises a threaded hole 114 at the center of the surface, that accepts screw 98. Surface 106 also comprises four substantially similar cylindrical protuberances 110 located at the corners of a non-square rectangle, and distributed symmetrically about hole 114. As described below, protuberances 110 act as connections for base 44. There is a cylindrical protuberance 112 that in the illustrated embodiment is centered on a longer side of the non-square rectangle, and that is larger than protuberances 110. Extending from base 44, and fixed to the base, is a divot 118 that acts as a verification point or indicator and that is herein also termed divot indicator 118 or just indicator 118. There is an aperture in surface 106 that provides access to screw 80.

FIG. 2C illustrates a lower surface 122 of socket 78, and an upper surface of the socket is shown in FIG. 2A. Lower surface 122 comprises four cylindrical recesses 126 that are dimensioned and positioned to accept protuberances 110 of surface 106, when socket 78 is placed on surface 106, so that recesses 126 also act as connections. An upper surface 126 of the socket comprises two access holes 134, distributed symmetrically about clearance hole 102. In addition, when socket 78 is mated with surface 106, clearance hole 102 aligns with threaded hole 114, so that screw 98 may be used to attach the socket, and thus the target, to base 44.

The description above, of target 48 being directly attached to surface 106, i.e., without adapter 40 being used, corresponds to a first configuration of marker 52. It will be understood that because the connection between the base and the marker has a rotational symmetry which is a rectangular rotational symmetry (i.e., of 180°), e.g., of recesses 126 and protuberances 110, there are two possible orientations of the target (e.g., target 48) or marker (e.g., marker 52) with respect to the anchoring device (e.g., clamp 34), when the target or marker is attached to the base (e.g., base 44) in this first configuration. A partially exploded view of each orientation is exemplified in FIG. 3A and FIG. 3C.

In some embodiments, the connection between the target or the marker and the base may not have a rotational symmetry and thus may allow only one single orientation with respect to the anchoring device or with respect or the anchoring device base. In some embodiments, the connection between the target or the marker and the base may have a square rotational symmetry (i.e., of 90°) which may allow up to four orientations with respect to the anchoring device or with respect to its base. Thus, the connection and connection elements may be defined and accordingly formed to allow various rotational symmetries with may allow various maximal orientations, correspondingly.

In some embodiments, the target-base, marker-base, target-adapter or marker-adapter connection rotational symmetry may be around an axis perpendicular to the plane of the upper surface of the base, such as surface 106, and located at its center, such as hole 114 of surface 106. Z axis of FIG. 2A is an example for such a connection rotational symmetry axis.

FIG. 2D illustrates an upper surface 140 of adapter 40. Adapter 40 is generally circular, but may be of other shapes (e.g., rectangular), and clearance hole 144 is located at the center of the adapter. Adapter 40 comprises four cylindrical holes 148 that are dimensioned and positioned to accept protuberances 110 of surface 106, when the adapter is placed on surface 106. Holes 148 are distributed symmetrically about central clearance hole 144, and it will be understood that holes 148 form a non-square rectangle. Holes 148 penetrate to a lower surface of adapter 40, and act as connections. To enable a single non-ambiguous placement of the adapter, the adapter also comprises a fifth cylindrical hole 152, that is dimensioned and positioned to accept protuberance 112 when holes 148 connect with, i.e., mate with, protuberances 110.

Four cylindrical protuberances 156 are formed on surface 140, and they are arranged to be congruent to protuberances 110, and thus lie in a non-square rectangle. Protuberances 156 are distributed symmetrically about central hole 144, and are located so that the non-square rectangle they form is orthogonal to the non-square rectangle formed by holes 148. As for protuberances 110, protuberances 156 act as connections.

According to some embodiments of the present disclosure and as shown in FIGS. 2A-2D, the target or marker connections or connection elements used to connect the target or marker to the base are also used to connect the target or marker to the adapter and in a substantially similar manner. Thus, the connection between the adapter and the target or the marker has the same connection rotational symmetry as the connection between the target or the marker and the base. Accordingly, the maximal number of additional different marker orientations which may be allowed by the adapter is equal to the maximal number of marker orientations which may be allowed by the rotational symmetry of the connection between the base and the target or marker.

According to some embodiments and as shown in FIGS. 2A-2D, the connection between the target or marker and the base and/or the connection between the target or marker and the adapter are such that the adjustment or change of an orientation of the target or marker with respect to the base and/or marker is performed by completely disconnecting the marker from the base or from the adapter, and reconnecting it to the base or the adapter in a different orientation with respect to the base and/or adapter.

In operation of marker 52 (illustrated in FIGS. 2A-2D) using adapter 40, the adapter is first placed on surface 106 of base 44 so that holes 148 mate with protuberances 110, and so that hole 152 mates with protuberance 112. It will be understood that for this mating there is only one possible orientation of the adapter with respect to base 44, and that in this orientation clearance hole 144 aligns with threaded hole 114 of the base.

Once adapter 40 has been placed on surface 106, socket 78 of target 48 may be mated with the upper surface of the adapter, by pushing recesses 126 of the socket onto protuberances 156. In some embodiments, the mating is facilitated by forming retaining shoulders 160, 164 on the surface of the adapter, symmetrically on either side of hole 144, the shoulders having internal walls that are separated by the width of socket 78, together with a clearance value. In some embodiments, retaining one or more shoulders such as retaining shoulders 160 of adapter 40 may limit the number of orientations (e.g., to two orientations, as in adapter 40, or to a single orientation) and determine the orientations in which a target or a marker, such as target 48 or patient marker 52, may connect to the adapter with respect to the base and/or the adapter. In some embodiments, where the connection or connection elements of the adapter replace the connection or connection elements of the base (e.g., forming the same or substantially the same connection with the marker as the base), one or more such retaining shoulders may be used to prevent the connection of a registration marker, which has a single non-ambiguous predefined location with respect to the anchoring device and/or base, to the adapter. Registration markers of this sort are described, for example, in PCT International Publication WO2021019369. The disclosures of this patent application publication is incorporated herein by reference.

The description above, of target 48 being attached to surface 106 via adapter 40, corresponds to a second configuration of target 48 or marker 52. It will be understood that in this second configuration, there are two possible orientations of target 48 with respect to clamp 34, when the target is attached to adapter 40. A partially exploded view of each orientation of the second configuration is shown in FIG. 3B and FIG. 3D.

First and second divots 168, 172, which act as verification points or indicators and which are also herein termed first and second divot indicators 168, 172, or just indicators 168, 172, are formed in adapter 40 as extensions of surface 140. First indicator 168 is positioned so that it aligns with, and prevents access to, indicator 118 when the adapter is attached to surface 106. Second indicator 172 is positioned so that the angle between a line segment from indicator 172 to central hole 144 and a line segment from indicator 168 to the central hole is an obtuse angle, less than 180°. In a disclosed embodiment the angle is approximately 160°, but other angles are possible. Positioning of second indicator 172 is referred to further below.

As stated above, marker 52 has two possible configurations, and for each configuration target 48 has two possible orientations with respect to clamp 34. There are thus four different orientations of target 48 with respect to the clamp. As is described below, using images of optical elements 86 of target 48, and being provided with the positions of indicators 118, 168, or 172, processor 26 is able to identify in which of the four orientations target 48 is. As is also described below, the processor is able to adjust the images presented to the professional in the augmented reality assembly to compensate for any change in the orientation. FIGS. 3A, 3B, 3C, and 3D illustrate the four possible orientations of target 48 with respect to clamp 34, according to an embodiment of the present disclosure. In each of the figures, marker 52 is drawn as a partially exploded view of the marker, and also as a top-down, i.e., down the z-axis, view of the marker. From consideration of the figures, and of the construction of the elements of marker 52 described above, it will be understood that in each of the four orientations target 48 is rotated clockwise by a multiple of 90° from the x-axis, i.e., in FIG. 3A by 0°, in FIG. 3B by 90°, in FIG. 3C by 180°, and in FIG. 3D by 270°. The orientations are also referred to herein as the 0°, 90°, 180°, and 270° orientation.

In the first configuration of the marker, i.e., when adapter 40 is not used, corresponding to FIGS. 3A and 3C and the 0° and 180° orientations, the only indicator present, and thus accessible, is indicator 118 of base 44.

In the second configuration of the marker, when adapter 40 is used, corresponding to FIGS. 3B and 3D and the 90° and 270° orientations, all three indicators 118, 168, and 172, are present. However, the indicators of adapter 40 are arranged so that only one of indicators 118, 168, and 172 is accessible in each orientation. Thus, in the 90° orientation illustrated in FIG. 3B, indicators 118 and 168 are obscured by connecting beam 82, and only indicator 172 is accessible. In the 270° orientation illustrated in FIG. 3D, indicator 172 is obscured by connecting beam 82, and indicator 168 obscures indicator 118 of base 44, so that only indicator 168 is accessible.

It will be appreciated that in each of the four possible orientations, only one indicator is accessible.

Using the locations of the accessible indicators, for each orientation processor 26 calculates a respective vector from a preselected point on target 48 to the accessible indicator. In the figures, the preselected point is assumed to be an optical element 86 at a corner of target 48, herein termed element 86A, but any other convenient preselected point on the target, such as a center of the target, may be used. As shown in the figures, for orientations 0°, 90°, 180°, and 270° there are respective vectors V1, V2, V3, and V4.

Table I below illustrates the orientations, vectors and parameters associated with the vectors. The vector parameters are assumed to comprise ordered triples, as measured with respect to the xyz axes described above.

TABLE I

| Orientation | Vector | Vector Parameters |
|---|---|---|
| 0° | V1 | (x₁, y₁, z₁) |
| 90° | V2 | (x₂, y₂, z₂) |
| 180° | V3 | (x₃, y₃, z₃) |
| 270° | V4 | (x₄, y₄, z₄) |

Table II below gives a numerical example, according to a disclosed embodiment, of approximate vector parameters, in mm, for the different orientations.

TABLE II

| Orientation | Vector | Vector Parameters |
|---|---|---|
| 0° | V1 | (−103, −42, −13) |
| 90° | V2 | (−17, +124, −8) |
| 180° | V3 | (+103, +8, −13) |
| 270° | V4 | (+25, −126, −8) |

Since the dimensions of all elements of marker 52 and adapter 44 are known, numerical values of the vectors of the different possible orientations may be precalculated and stored by processor 26. The vectors may be stored in any convenient format known in the art, for example related to a cylindrical coordinate system, or to a spherical coordinate system, or to another Cartesian set of axes, rather than the Cartesian axes presented here. All such formats are assumed to be comprised within the scope of the present disclosure.

FIG. 4 is a flowchart describing the use of a marker, such as marker 52 and/or and adapter, such as adapter 40, in the medical procedure referred to above and a computerized method for updating a registration between a patient marker and a patient due to a change in the orientation of the patient marker during a medical procedure, according to an embodiment of the present disclosure.

In an initial step 150, professional 22 attaches clamp 34 or any other anchoring device to a bone of patient 30, herein assumed to comprise a spinous process of the patient, by rotating screw 80. A registration marker is attached to the clamp and the clamp and/or the registration marker is registered to the patient, typically by a CT (computerized tomography) scan. A patient marker, such as patient marker 52, is attached to the clamp in a location with respect to the registration marker which may be predetermined or tracked and calculated. Methods for registering an anchoring device such as a clamp and/or a registration marker and thus a patient marker to a patient is provided in PCT International Publication WO2021019369 incorporated by reference hereinabove and U.S. Patent Application Publication No. 2021-0161614, which is incorporated herein by reference. In the initial step, processor 26 registers the clamp and/or patient marker to the patient and stores the preselected point on the target (referred to above) and distinct orientation value for each marker orientation (e.g., four distinct orientation values for four different marker orientation). The orientation value is determined based on a position of an indicator, associated with the marker orientation, with respect to the marker (e.g., with respect to the preselected point on the target). The orientation value may be a three-dimensional location vector in a predetermined coordinate system (e.g., a coordinate system of the anchoring device or a coordinate system of the marker) determined by vector parameters such the vectors and vector parameters for the four orientations of marker 52, corresponding to the values in Table I.

In a target attachment step 154, the professional uses screw 98 to attach alignment target 48 or patient marker 52 to the clamp, in one of the four orientations illustrated in FIGS. 3A-3D. It will be understood that depending on the orientation selected, the professional may or may not use adapter 40 for the attachment.

Once alignment target 48 is attached to the clamp, camera 68 and projector 69 are activated, so that processor 26 begins to track the alignment target and the preselected point in the target.

In a tool activation step 158, professional 22 introduces tool 46 into proximity with the patient, and initiates tracking of the tool. The tracking uses tool marker 50, and images of the marker generated in camera 68 from radiation of projector 69. The tracking provides processor 26 with the position of tool tip 54. In some embodiments, processor 26 performs the tracking of tool 54 via camera 68.

In an indicator access step 162, professional 22 touches tool tip 54 on the single indicator, i.e., indicator 118, 168, or 172, that is accessible by virtue of the orientation of target 48 formed in step 154. Processor 26 may recognize that the indicator is being touched by any convenient method known in the art, for example by the tool tip being held on, or in proximity to, the indicator for a preselected period of time, and/or by the tool tip being within a preselected region of the tracked alignment target.

In an orientation calculation step 166, from coordinates of the accessible indicator position and of the preselected point position in the tracked alignment target the processor calculates the orientation value, e.g., coordinates of the vector parameters (as in Table I) joining the positions, and from the parameters stored in step 150, identifies the orientation of step 154.

Step 166 completes an initial setup set of steps of the flowchart. The remaining steps are performed as the procedure being performed by professional 26 continues.

Control in the flowchart passes to a decision step 170, where the processor iteratively checks if a change of orientation of the alignment target has occurred. Typically, the change is effected by professional 26 removing then re-attaching the target to clamp 34.

The processor may perform its check by issuing a request, after a preset time period has passed, to the professional to touch tool tip 54 on the single accessible indicator. Alternatively or additionally the professional may inform the processor that a change has been made by any convenient method known in the art, for example by placing tool tip 54 on the single accessible indicator for the preselected period of time referred to above. Alternatively, or additionally the processor may compute a current orientation value, e.g., location vector parameters for the current location of the tool tip with respect to the marker in each image captured by camera 68, or in each preselected number of images, and compare these parameters to the stored parameters to identify if a change of orientation has been made. A stored orientation value will be received for a calculated current orientation value only if the tool tip is located on the indicator associated with the current marker orientation. Once such a stored orientation value is received, the processor may check if this value is different from the previously received stored orientation value which corresponds to the preceding marker orientation. The preceding marker orientation may be the same or different. In some embodiments, a change of orientation is identified based on a sequence of a predefined number of images exhibiting such a change.

If decision step 170 returns negative, i.e., the processor finds there is no change in the target orientation, then in a no change step 174, the processor continues using the existing vector, and control returns to decision step 170.

If decision step 170 returns positive, i.e., the processor finds there is a change in the target orientation then in a change vector step 178 the processor calculates the new coordinates of the new target vector, as described in step 166. The processor uses the new vector to determine the new orientation of the target, and uses the new orientation to continue tracking the target.

In an update registration step 182, the processor uses the new orientation to update the registration of clamp 34 with patient 30. Control then returns to decision step 170.

FIG. 5A is a schematic illustration of a head-mounted display (HMD) 200 in the form of spectacles or glasses, according to an embodiment of the present disclosure. HMD 200 is worn by professional 22, and is similar to and may be used in place of assembly 24 (FIG. 1).

Typically, head-mounted device 200 includes a tracking device 230 that is configured to facilitate determination of the location and orientation of head-mounted device 200 with respect to marker 48 (or patient 30) and/or with respect to tool 46.

Tracking device 230 includes an image-capturing device 208, typically a camera and herein referred to as camera 208, that is configured to image reflective elements 86 (shown in FIG. 2A) of patient marker 52 and reflective elements of tool marker 50. Patient marker 52 and tool marker 50 act as identifying markers for respective entities associated with the markers.

Tracking device 230 includes a light source 216, which is mounted on HMD 200. The light source is typically configured to irradiate the patient marker and the tool marker, such that light reflects from marker reflective elements toward camera 208. For example, light source 216 may be an infrared light source (for example, a light source that generates light at a wavelength of between 700 nm and 800 nm) and the camera may include a corresponding infrared filter. Optionally, HMD 200 may include additional one or more cameras 250, which are configured to capture images of scenes in the visible spectrum.

HMD 200 further comprises a pair of augmented reality displays 220, which allow professional 22 to view entities, such as part or all of patient 30 through the displays, and which are also configured to present to the professional images that may be received from database 38, camera 250 or may present any other information. HMD 200 comprises a frame 280 which, inter alia, holds displays 220 and temples 260. Frame 280 comprises a nose bridge 270.

HMD also includes a processing unit of processor 26 (not shown), which operates elements of the HMD. Alternatively, HMD 200 may comprise processor 26. The processing unit or processor 26 may be mounted on or integrated within frame 280 or temples 260. In some embodiments, the processing unit typically may communicate with other processing units of processor 26, e.g., via an antenna (not shown).

Elements of the HMD are typically powered by a battery (not shown in the figure). HMD 200 is held in place on the head of professional 22 by temples 260 and nose bridge 270.

FIG. 5B is a schematic figure illustrating a head-up display (HUD) or head-mounted display (HMD) 300, according to another embodiment of the present disclosure. HMD 300 is worn by professional 22, and may be used in place of assembly 24 (FIG. 1). HMD 300 comprises an optics housing 304 which incorporates an infrared camera 308. Housing 304 also comprises an infrared transparent window 312, and within the housing, i.e., behind the window, are mounted one or more infrared projectors 316. Mounted on housing 304 are a pair of augmented reality displays 320, which allow professional 22 to view entities, such as part or all of patient 30 through the displays, and which are also configured to present to the professional images that may be received from database 38 or any other information.

The HUD includes a processing unit 324 (or a processing unit of processor 26), mounted in a processor housing 326, which operates elements of the HMD. In some embodiments, processor 324 may communicate with other processing units of processor 26 via an antenna 328.

Mounted on the front of HMD 300 is a flashlight 332. The flashlight projects visible spectrum light onto objects so that professional 22 is able to clearly see the objects through displays 320. Elements of the head-up display are typically powered by a battery (not shown in the figure) which supplies power to the elements via a battery cable input 336.

HUD 300 is held in place on the head of professional 22 by a head strap 340, and the professional may adjust the head strap by an adjustment knob 344.

It will be understood that although the above description specifically refers to a group of discrete orientations comprising the angles of 0°, 90°, 180°, and 270°, those having ordinary skill in the art will be able to adapt the description, mutatis mutandis, for other groups of discrete orientations, such as 0°, 60°, 120°, and 180°, or other groups comprising number of orientation different than four, such as 0°, 120°, 240° or 0°, 60°, 120°, 180°, 240° and 300°, and all such groups are considered to be comprised within the scope of the present disclosure.

It will be understood that although the above description specifically refers to embodiments utilizing three indicators, while the first is comprised in the anchoring device base and the other two are comprised in the adapter, those having ordinary skill in the art will be able to adapt this description, mutatis mutandis, for other embodiments comprising a different number of indicators, or a different arrangement of indicators. For example, in some embodiments only two indicators, one comprised in the base and the other comprised in the adaptor, may be utilized. Each such configuration comprises a group of marker orientations comprising two or more distinct marker orientations and a group of indicators comprising one or more indicators. Each marker orientation is associated with an indicator from the group of indicators. In each marker orientation, only the associated indicator should be accessible to prevent confusion and facilitate simple and non-ambiguous determination of orientation. Each such pair of marker orientation and associated or corresponding indicator should provide a distinct location vector, e.g., determining the location of the indicator relative to the marker in a predefined coordinate system.

While the description above assumes that anchoring device 34 comprises a clamp, it will be understood that the anchoring device may comprise other types of attachment to the bone of patient 30, such as a pin that is inserted into the bone. Such a pin and a pin adapter are disclosed, for example, in PCT International Application IB2022/056212 incorporated by reference hereinabove, while the marker or target or the marker adapter of the present disclosure may be attached to the pin via the pin adapter. Thus, the scope of the present disclosure includes all such types of attachment.

In accordance with several embodiments, processor 26 typically comprises a general-purpose computer processor, which is programmed in software to carry out one or more of the functions described hereinabove and such as registration, tracking, image analysis, orientation values calculation. This software (e.g., executable program instructions) may be stored on tangible, non-transitory computer-readable media, such as optical, magnetic, or electronic memory media. Additionally or alternatively, at least some of the functions of processor 26 may be carried out using special-purpose computing hardware, such as a graphics processing unit (GPU).

It will be appreciated that the embodiments described above are cited by way of example, and that the present disclosure is not limited to what has been particularly shown and described hereinabove. Rather, the scope of the present disclosure includes both combinations and subcombinations of the various features described hereinabove, as well as variations and modifications thereof which would occur to persons skilled in the art upon reading the foregoing description and which are not disclosed in the prior art.

1.-14. (canceled)

15. A method of updating a registration associated with a moveable patient marker used in a medical procedure, wherein:
the patient marker is coupled to an anchoring device via an adapter,
the adapter is coupled to the anchoring device via a base,
the anchoring device is attached to a patient, and
the base comprises at least one base indicator, and the adapter comprises at least one adapter indicator,
the method comprising, by at least one processor:
registering the patient marker to the patient;
storing a plurality of distinct orientation values, each orientation value corresponds to a different orientation of the patient marker with respect to the anchoring device and associated with an indicator, wherein the indicator is the at least one base indicator or the at least one adapter indicator, and wherein the orientation value indicates a position of the indicator associated with the orientation value with respect to the patient marker;
tracking the position of a tool marker with respect to the patient marker by capturing images of the patient marker and the tool marker via a camera, wherein the tool marker is attached to a tool used in the medical procedure;
determining a current marker orientation value based on analysis of one or more images of the patient marker and the tool marker;
repeatedly calculating subsequent marker orientation values for images captured by the camera; and
if a change in marker orientation value was identified in the subsequent marker orientations with respect to the current marker orientation value, then a change in the patient marker orientation is determined and the registration of the patient marker to the patient is updated accordingly.

16. The method of claim 15, wherein in any orientation of the patient marker with respect to the anchoring device, only one of the at least one base indicator or the at least one adapter indicator is accessible.

17. The method of claim 15, wherein the base comprises base connections, the adapter comprises adapter connections, and the patient marker comprises marker connections, and wherein the base connections are used to connect the base to the patient marker or to the adapter, and the marker connections are used to connect the patient marker to the base or to the adapter.

18. (canceled)

19. (canceled)

20. (canceled)

21. A method of updating a registration associated with a movable patient marker used in a medical procedure, wherein:
the patient marker is coupled to an anchoring device via an adapter,
the adapter is coupled to the anchoring device via a base,
the anchoring device is attached to a patient,
the base comprises at least one base indicator,
the adapter comprises at least one adapter indicator, and
a tool for use in the medical procedure comprises a tool marker,
the method comprising, by at least one processor:
tracking a position of the tool marker with respect to the patient marker by analyzing images, captured by at least one camera, of the patient marker and the tool marker;
detecting that a tip of the tool is in proximity to an accessible indicator of the at least one base indicator or the at least one adapter indicator;
calculating a vector between a preselected point of the patient marker and the accessible indicator;
determining, using the calculated vector, a current patient marker orientation with respect to the anchoring device, by referencing one or more stored vector parameters, wherein the stored vector parameters each correspond to one of a plurality of discrete patient marker orientations with respect to the anchoring device;
checking whether the determined current patient marker orientation is indicative of a change in patient marker orientation; and
responsive to determining that a change in patient marker orientation has occurred, updating the registration to correspond to the current patient marker orientation.

22. The method of claim 21, wherein in any of the plurality of discrete patent marker orientations with respect to the anchoring device, only one of the at least one base indicator or the at least one adapter indicator is accessible.

23. The method of claim 21, wherein the base comprises base connections, the adapter comprises adapter connections, and the patient marker comprises marker connections, and wherein the base connections are used to connect the base to the patient marker or to the adapter, and the marker connections are used to connect the patient marker to the base or to the adapter.

24. The method of claim 21, wherein the detecting that the tip of the tool is in proximity to the accessible indicator comprises determining that the tip of the tool has been held on the accessible indicator for a preselected period of time.

25. The method of claim 21, wherein the detecting that the tip of the tool is in proximity to the accessible indicator comprises determining that the tip of the tool has been held in proximity to the accessible indicator for a preselected period of time.

26. The method of claim 21, wherein the detecting that the tip of the tool is in proximity to the accessible indicator comprises determining that the tip of the tool is within a preselected region.

27. The method of claim 21, further comprising repeating the calculating, determining, and checking, responsive to detecting that the tip of the tool is again in proximity to an accessible indicator of the at least one base indicator or the at least one adapter indicator.

28. The method of claim 27, further comprising, responsive to determining that another change in patient marker orientation has occurred, updating the registration to correspond to a new current patient marker orientation.

29. The method of claim 21, wherein the anchoring device comprises a clamp.

30. The method of claim 21, wherein the anchoring device comprises a pin.

31. The method of claim 15, wherein the determining a current marker orientation value is performed responsive to detecting that a tip of the tool is in proximity to an accessible indicator of the at least one base indicator or the at least one adapter indicator.

32. The method of claim 31, wherein the detecting that the tip of the tool is in proximity to the accessible indicator comprises determining that the tip of the tool has been held on the accessible indicator for a preselected period of time.

33. The method of claim 31, wherein the detecting that the tip of the tool is in proximity to the accessible indicator comprises determining that the tip of the tool has been held in proximity to the accessible indicator for a preselected period of time.

34. The method of claim 31, wherein the detecting that the tip of the tool is in proximity to the accessible indicator comprises determining that the tip of the tool is within a preselected region.

35. The method of claim 15, wherein the anchoring device comprises a clamp.

36. The method of claim 15, wherein the anchoring device comprises a pin.
